(19) United States
(12) Patent Application Publication
(10) Pub. No.: US 2021/0393167 A1
(43) Pub. Date: Dec. 23, 2021

(54) POSTURE IMPROVEMENT ASSISTANCE DEVICE, POSTURE IMPROVEMENT ASSISTANCE METHOD, AND NON-TRANSITORY STORAGE MEDIUM STORING POSTURE IMPROVEMENT ASSISTANCE PROGRAM

(71) Applicant: OMRON HEALTHCARE Co., Ltd., Kyoto (JP)

(72) Inventors: Tamaki ITO, Kyoto (JP); Mitsuru SAMEJIMA, Kyoto (JP); Keisuke YAMADA, Kyoto (JP)

(21) Appl. No.: 17/447,148

(22) Filed: Sep. 8, 2021

Related U.S. Application Data
(63) Continuation of application No. PCT/JP2020/006645, filed on Feb. 19, 2020.

(30) Foreign Application Priority Data
Mar. 12, 2019 (JP) 2019-044981

(51) Int. Cl. A61B 5/11; A61B 5/00

(52) U.S. Cl. CPC A61B 5/1116; A61B 5/742; A61B 2562/0219; A61B 5/6823; A61B 5/7405

(57) ABSTRACT

The present invention provides a posture improvement assistance device, a posture improvement assistance method, and a non-transitory storage medium storing a posture improvement assistance program that give a user a strong motivation to improve one's posture and assist in achieving a good posture. A control unit of an electronic device includes a posture acquisition unit (21A) that acquires information on a posture of a user who is using a display unit; and a display control unit that, when the posture satisfies a condition, controls a display condition of an image displayed on the display unit to be in a reference state, and when the posture does not satisfy the condition, controls the display condition to be in a state that is changed with respect to the reference state.

### CROSS-REFERENCE TO RELATED APPLICATIONS

This application is the U.S. national stage application filed pursuant to 35 U.S.C. 365(c) and 120 as a continuation of International Patent Application No. PCT/JP2020/006645, filed Feb. 19, 2020, which application claims priority to Japanese Patent Application No. 2019-044981, filed Mar. 19, 2019, which applications are incorporated herein by reference in their entireties.

### TECHNICAL FIELD

The present invention relates to a posture improvement assistance device, a posture improvement assistance method, and a non-transitory storage medium storing a posture improvement assistance program.

### BACKGROUND ART

Having proper posture when seated, such as during desk work or watching a video, or when moving, such as during jogging or walking, is important in reducing a bodily load and in improving exercise performance. Patent Documents 1 to 3 describe systems that support achieving proper posture by detecting a person's posture with a sensor attached to the person and giving the person a notification.

### CITATION LIST

### Patent Literature

Patent Document 1: JP 2011-240047 A
Patent Document 2: JP 2015-058096 A
Patent Document 3: JP 2018-082770 A

### SUMMARY OF INVENTION

### Technical Problem

If a user's posture deviates from the proper posture, that status must be effectively conveyed to the user in order for the user to achieve a good posture. Patent Documents 1 and 2 describe notifying a user by issuing a notification sound when a posture is not a desired posture. However, simply issuing a notification sound provides little motivation for the user to improve one's posture. In Patent Document 3, a posture model that models the user's movement is displayed in order to make a user aware of one's posture. However, simply showing the posture model provides little motivation for the user to improve one's posture.

An object of the present invention is to provide a posture improvement assistance device, a posture improvement assistance method, and a posture improvement assistance program that give a user a strong motivation to improve one's posture and assist in achieving good posture.

### Solution to Problem

(1) A posture improvement assistance device (for example, a control unit 21 or a control unit 31 according to embodiments described below) includes: a posture acquisition unit that acquires information on a posture of a user who is looking at an image displayed on a display unit of an electronic device or a user who is listening to sound output from an audio output unit of an electronic device; and a control unit (for example, a display control unit 21B or an audio output control unit 31B according to embodiments described below) that, when the information on the posture of the user satisfies a condition that is predetermined and represents a state of proper posture, controls a display condition of the image displayed on the display unit or an output condition of the sound being output from the audio output unit to be in a reference state, and when the information on the posture of the user does not satisfy the condition, controls the display condition or the output condition to be in a state that is changed with respect to the reference state.

According to (1), in a state where the user's posture does not satisfy the condition, the display condition of the image or the output condition of the sound is controlled to change from the reference state to a changed state, and therefore the user viewing the image or listening to the audio can be made uncomfortable, for example. Therefore, to eliminate this discomfort, the user can be expected to improve one's posture naturally, and the user can be given a strong motivation to improve one's posture. Further, the change from the reference state allows the user to intuitively understand the need to improve one's posture, and also the user can achieve a state in which one's posture satisfies the condition simply by changing one's posture such that the display condition of the image or the output condition of the sound is restored to the reference state.

(2) The posture improvement assistance device according to (1), wherein the display condition relates to a display position of the image, and, when the information on the posture of the user satisfies the condition, the control unit controls the display position of the image displayed on the display unit to be in the reference state as a reference position, and when the information on the posture of the user does not satisfy the condition, the control unit changes the display position of the image to a position different from the reference position.

According to (2), in a case where the user's posture does not satisfy the condition, the display position of the image displayed on the display unit is changed to a position different from the reference position, in comparison to a case where the user's posture satisfies the condition. Therefore, if the reference position is, for example, a position set by the user, discomfort due to the display position of the image deviating from the reference position can be conveyed to the user, and the user can be strongly expected to improve one's posture in order to eliminate this discomfort.

(3) The posture improvement assistance device according to (1), wherein the display condition relates to a display size of the image, and, when the information on the posture of the user satisfies the condition, the control unit controls the display size of the image displayed on the display unit to be in the reference state as a reference size, and when the information on the posture of the user does not satisfy the condition, the control unit changes the display size of the image to a size different from the reference size.

According to (3), in a case where the user's posture does not satisfy the condition, the display size of the image displayed on the display unit is changed to a size different from the reference size, in comparison to a case where the user's posture satisfies the condition. Therefore, if the reference size is, for example, a size set by the user, discomfort due to the display size of the image being changed from the reference size can be conveyed to the user, and the user can be strongly expected to improve one's posture in order to eliminate this discomfort.

(4) The posture improvement assistance device according to (1), wherein the display condition relates to a display posture of the image, and, when the information on the posture of the user satisfies the condition, the control unit controls the display posture of the image displayed on the display unit to be in the reference state as a reference posture, and when the information on the posture of the user does not satisfy the condition, the control unit changes the display posture of the image to a posture rotated from the reference posture.

According to (4), in a case where the user's posture does not satisfy the condition, the display posture of the image displayed on the display unit is changed to a state rotated from the reference posture, in comparison to a case where the user's posture satisfies the condition. Therefore, if the reference posture is, for example, a posture set by the user, discomfort due to the display posture of the image being changed from the reference posture can be conveyed to the user, and the user can be strongly expected to improve one's posture in order to eliminate this discomfort.

(5) The posture improvement assistance device according to (1), wherein the display condition relates to a display shape of the image, and, when the information on the posture of the user satisfies the condition, the control unit controls the display shape of the image displayed on the display unit to be in the reference state as a reference shape, and when the information on the posture of the user does not satisfy the condition, the control unit changes the display shape of the image to a shape different from the reference shape.

According to (5), in a case where the user's posture does not satisfy the condition, the shape of the image displayed on the display unit is changed from the reference shape, in comparison to a case where the user's posture satisfies the condition. Therefore, if the reference shape is, for example, a shape set by the user, discomfort due to the shape of the image being changed from the reference shape can be conveyed to the user, and the user can be strongly expected to improve one's posture in order to eliminate this discomfort.

(6) The posture improvement assistance device according to (1), wherein the audio output unit includes a first audio output unit and a second audio output unit, the output condition relates to a difference or a ratio between a volume of the sound output from the first audio output unit and a volume of the sound output from the second audio output unit, and, when the information on the posture of the user satisfies the condition, the control unit controls the difference or the ratio to be in the reference state as a reference value, and when the information on the posture of the user does not satisfy the condition, the control unit controls the difference or the ratio to be a value different from the reference value.

According to (6), in a case where the user's posture does not satisfy the condition, the balance of the sound being output from the audio output unit can be disrupted, in comparison to a case where the user's posture satisfies the condition. Therefore, discomfort due to the output balance of the sound being changed can be conveyed to the user, and the user can be strongly expected to improve one's posture in order to eliminate this discomfort.

(7) The posture improvement assistance device according to (1), wherein the sound is music that includes audio from a plurality of audio sources, the output condition relates to a balance of a volume of the audio for each of the audio sources included in the music output from the audio output unit, and when the information on the posture of the user satisfies the condition, the control unit controls the audio from each of the plurality of audio sources to be in the reference state, the audio being output from the audio output unit at a volume that is predetermined, and when the information on the posture of the user does not satisfy the condition, the control unit changes only the volume of the audio from an audio source, which is a portion of the plurality of audio sources.

According to (7), in a case where the user's posture does not satisfy the condition, only audio from a specific audio source being output from the audio output unit will sound too loud or too soft, in comparison to a case where the user's posture satisfies the condition. Therefore, discomfort due to this increase or decrease in the audio can be conveyed to the user, and the user can be strongly expected to improve one's posture in order to eliminate this discomfort.

(8) The posture improvement assistance device according to (7), wherein, when the information on the posture of the user does not satisfy the condition, the control unit only changes the volume of the audio from the audio source, which is the portion, to zero.

According to (8), in a case where the user's posture does not satisfy the condition, a portion of the sound being output from the audio output unit will become inaudible, in comparison to a case where the user's posture satisfies the condition. Therefore, discomfort can be conveyed to the user due to the disruption of audio balance from the lack of audio, and the user can be strongly expected to improve one's posture in order to eliminate this discomfort.

(9) The posture improvement assistance device according to (7) wherein, when the information on the posture of the user does not satisfy the condition, the control unit increases only the volume of the audio from the audio source, which is the portion.

According to (9), in a case where the user's posture does not satisfy the condition, a portion of the sound being output from the audio output unit becomes louder, in comparison to a case where the user's posture satisfies the condition. Therefore, discomfort can be conveyed to the user due to the disruption of audio balance from the increase in audio, and the user can be strongly expected to improve one's posture in order to eliminate this discomfort.

(10) The posture improvement assistance device according to (8) or (9), wherein the control unit changes the audio source, which is the portion, in accordance with a direction of bias in a center of gravity of the user based on the information on the posture of the user.

According to (10), the user can intuitively recognize what to do so that one's posture satisfies the condition.

(11) A posture improvement assistance method includes: a posture acquisition step acquiring information on a posture of a user who is looking at an image displayed on a display unit of an electronic device or a user who is listening to audio output from an audio output unit of an electronic device; and a control step, when the information on the posture of the user satisfies a condition that is predetermined and represents a state of proper posture, controlling a display condition of the image displayed on the display unit or an output condition of the sound being output from the audio output unit to be in a reference state, and when the information on the posture of the user does not satisfy the condition, controlling the display condition or the output condition to be in a state that is changed with respect to the reference state.

(12) A non-transitory storage medium storing a posture improvement assistance program, the program causing a computer to execute: a posture acquisition step acquiring information on a posture of a user who is looking at an image displayed on a display unit of an electronic device or a user who is listening to audio output from an audio output unit of an electronic device; and a control step, when the information on the posture of the user satisfies a condition that is predetermined and represents a state of proper posture, controlling a display condition of the image displayed on the display unit or an output condition of the sound being output from the audio output unit to be in a reference state, and when the information on the posture of the user does not satisfy the condition, controlling the display condition or the output condition to be in a state that is changed with respect to the reference state.

### Advantageous Effects of Invention

According to the present invention, a posture improvement assistance device, a posture improvement assistance method, and a posture improvement assistance program can be provided that give a user a strong motivation to improve one's posture and assist in achieving good posture.

### BRIEF DESCRIPTION OF DRAWINGS

FIG. 1 is a schematic diagram illustrating a schematic configuration of a posture improvement assistance system 100.

FIG. 2 is a diagram illustrating functional blocks of a control unit 21 of the posture improvement assistance system 100 illustrated in FIG. 1.

FIG. 3 is a diagram schematically illustrating a display unit 22 illustrated in FIG. 1.

FIG. 4 is a diagram illustrating variations of a display form of an image displayed on the display unit 22.

FIG. 5 is a diagram illustrating other variations of the display form of the image displayed on the display unit 22.

FIG. 6 is a diagram illustrating still other variations of the display form of the image displayed on the display unit 22.

FIG. 7 is a schematic diagram illustrating a schematic configuration of a posture improvement assistance system 200, which is a modified example of the posture improvement assistance system 100 illustrated in FIG. 1.

FIG. 8 is a diagram illustrating functional blocks of a control unit 31 of the posture improvement assistance system 200 illustrated in FIG. 7.

FIG. 9 is a schematic diagram for explaining the content of changes to an output condition made by an audio output control unit 31B illustrated in FIG. 8.

FIG. 10 is a schematic diagram for explaining another example of the content of changes to the output condition made by the audio output control unit 31B illustrated in FIG. 8.

FIG. 11 is a schematic diagram for explaining yet another example of the content of changes to the output condition made by the audio output control unit 31B illustrated in FIG. 8.

### DESCRIPTION OF EMBODIMENTS

### Overview of a Posture Improvement Assistance System According to an Embodiment

A posture improvement assistance system according to an embodiment provides assistance such that a user achieves a good posture (hereinafter referred to as an attractive posture) that applies little load on the body. A posture improvement assistance device included in the posture improvement assistance system determines the posture of the user based on output information from a motion detection sensor attached to the user or a captured image of the user, for example. Then, in a case where the determined posture of the user satisfies a condition for the attractive posture, the posture improvement assistance device displays an image in a normal display form on a display unit that the user is observing.

On the other hand, in a case where the determined posture of the user does not satisfy the condition for the attractive posture, the posture improvement assistance device changes, for example, the position, size, inclination, or shape of the image displayed on the display unit that the user is observing. For example, when the user's posture deviates from the attractive posture, changes occur in the display form of an image displayed in the center of the display unit, such as the image moving to the left or right, the image rotating, the image becoming larger or smaller, or the shape of the image changing from a rectangle to a rhombus. This may make the user viewing the image feel uncomfortable. Therefore, to eliminate this discomfort, the user can be expected to try to improve one's posture naturally, and the user can be given a strong motivation to improve one's posture.

### Specific Configuration of the Posture Improvement Assistance System According to the Embodiment

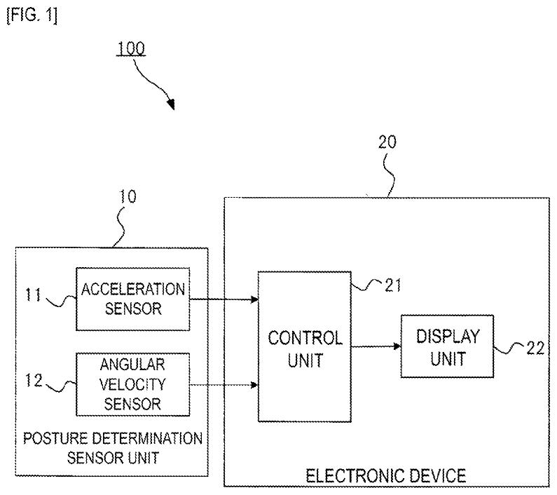

FIG. 1 is a schematic diagram illustrating a schematic configuration of a posture improvement assistance system 100. The posture improvement assistance system 100 illustrated in FIG. 1 includes a posture determination sensor unit 10 and an electronic device 20.

The posture determination sensor unit 10 is used by being attached to the body (e.g., the waist) of a user using the electronic device 20 and outputs information related to user movement (e.g., movement of the body's center of gravity) that is required in order to determine the user's posture.

In the example of FIG. 1, the posture determination sensor unit 10 includes an acceleration sensor 11 and an angular velocity sensor 12. Information detected by the acceleration sensor 11 and information detected by the angular velocity sensor 12 are transmitted to the electronic device 20 via a communication interface (not included in the drawings).

The electronic device 20 is, for example, a smartphone, a tablet-style terminal, a personal computer, or a television. Specifically, the electronic device 20 includes a control unit 21, a display unit 22, and a communication interface (not included in the drawings) for connecting to the posture determination sensor unit 10.

The control unit 21 includes, for example, a central processing unit (CPU), a random-access memory (RAM), and a read-only memory (ROM) and performs, for example, integrated control of the electronic device 20 in accordance with a program. The ROM of the control unit 21 stores a program that includes a posture improvement assistance program.

The display unit 22 is a device capable of displaying an image, such as a liquid crystal display panel or an organic electro-luminescence (EL) display panel. Note that the display unit 22 need not be installed in the electronic device 20 and may be provided outside the electronic device 20 so long as the display unit 22 can be connected to the control unit 21.

### Functions of the Control Unit 21

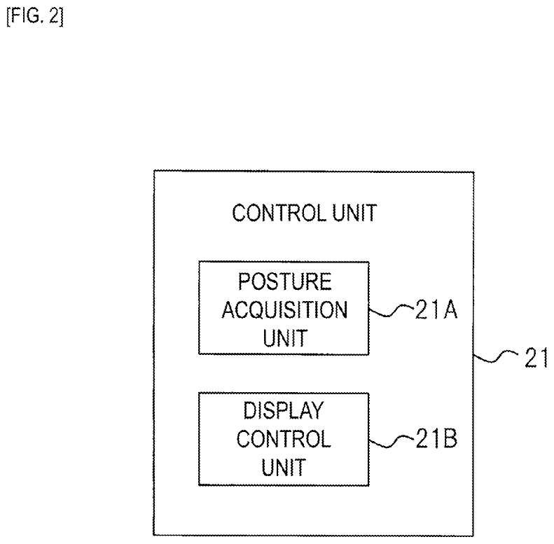

FIG. 2 is a diagram illustrating functional blocks of the control unit 21 of the posture improvement assistance system 100 illustrated in FIG. 1. The CPU of the control unit 21 functions as a posture acquisition unit 21A and a display control unit 21B by executing the above-described posture improvement assistance program stored in the ROM.

On the basis of the detection information received from the acceleration sensor 11 and the angular velocity sensor 12 of the posture determination sensor unit 10, the posture acquisition unit 21A determines the posture of the user wearing the posture determination sensor unit 10 and so acquires information on the user's posture.

Note that in a case where the user's posture is determined to be a seated posture, for example, the seated posture of the user can be determined by providing an imaging unit capable of capturing an image of a seated user, the posture acquisition unit 21A acquiring the image of the user captured by the imaging unit, the posture acquisition unit 21A analyzing the captured image. In addition, the posture determination sensor unit 10 may be provided with a processor that determines the posture of the user based on the captured image described above or based on the detection information from the acceleration sensor 11 and the angular velocity sensor 12, and may be configured to have the posture acquisition unit 21A acquire the information for the posture determined at this point.

The display control unit 21B determines whether the user's posture as acquired by the posture acquisition unit 21A satisfies a predetermined condition (a condition signifying the attractive posture described above) and controls a display condition for the image displayed on the display unit 22 on the basis of the determination result.

A “condition signifying the attractive posture” refers to, for example, one or both of: the amount of bias in the user's center of gravity to the left-right direction being less than or equal to a predetermined first threshold value (preferably zero) and the amount of bias in the user's center of gravity to the front-back direction being less than or equal to a predetermined second threshold value (preferably zero).

A “display condition for the image displayed on the display unit 22” refers to, for example, a display position of the image, a display size of the image, a display shape of the image, or a display posture of the image.

In the electronic device 20, a screen unrelated to the posture improvement assistance program can be displayed on the display unit 22 even in a state where the CPU of the control unit 21 is executing the posture improvement assistance program. Specifically, after attaching the posture determination sensor unit 10 to the user's body and executing the posture improvement assistance program on the electronic device 20, the user can execute another application program stored in the ROM of the control unit 21 to, for example, view a photo on the display unit 22, watch a video on the display unit 22, or create a document on the display unit 22.

In a case where the user's posture as acquired by the posture acquisition unit 21A satisfies the above-noted condition, the display control unit 21B displays an image displayed by such other application program on the display unit 22, with a display condition determined by that application program (the display condition in this case is defined as a reference state), and in a case where the user's posture does not satisfy the above-noted condition, the display control unit 21B displays the image with a display condition different from the display condition determined by that application program.

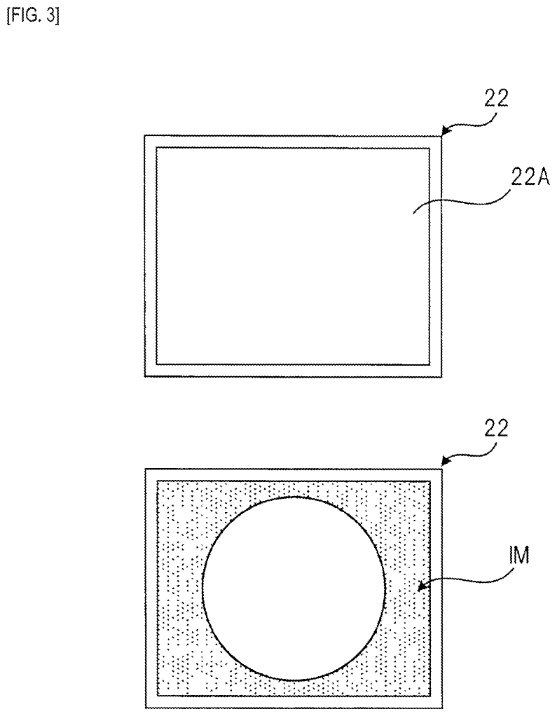

FIG. 3 is a diagram schematically illustrating the display unit 22 illustrated in FIG. 1. The display unit 22 includes a rectangular display area 22A. The bottom segment of FIG. 3 illustrates an example in which an image IM is displayed in the entire display area 22A.

### First Example of Changing Image Display Condition

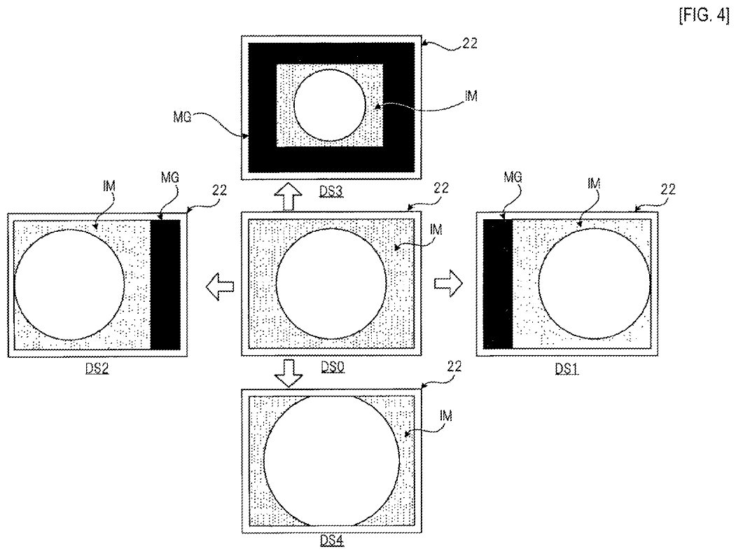

FIG. 4 is a diagram illustrating variations of the display form of the image displayed on the display unit 22. A display form DS0 illustrated in FIG. 4 illustrates a display form in a case where the user's posture satisfies the condition for the attractive posture. In a case where the user's posture satisfies the condition for the attractive posture, the image IM is displayed at a position (reference position) determined by the above-noted other application program, as illustrated by the display form DS0.

A display form DS1, a display form DS2, a display form DS3, and a display form DS4 illustrated in FIG. 4 each illustrate a display form for a case where the user's posture does not satisfy the condition for the attractive posture.

The display form DS1 illustrates a display form in a case where the amount of bias in the user's center of gravity to the right direction exceeds the first threshold value. In the display form DS1, the image IM is moved in the right direction relative to the display form DS0, and a non-display area MG of the image is formed in the display area 22A vacated by this movement.

The display form DS2 illustrates a display form in a case where the amount of bias in the user's center of gravity to the left direction exceeds the first threshold value. In the display form DS2, the image IM is moved in the left direction relative to the display form DS0, and the non-display area MG of the image is formed in the display area 22A vacated by this movement.

The display form DS3 illustrates a display form in a case where the amount of bias in the user's center of gravity to the front direction exceeds the second threshold value. In the display form DS3, the image IM is displayed at a reduced scale relative to the display form DS0, and the non-display area MG of the image is formed in the display area 22A vacated by this reduction.

The display form DS4 illustrates a display form in a case where the amount of bias in the user's center of gravity to the back direction exceeds the second threshold value. In the display form DS4, the image IM is displayed enlarged relative to the display form DS0, resulting in a state where checking the entire image IM is not possible.

### Second Example of Changing Image Display Condition

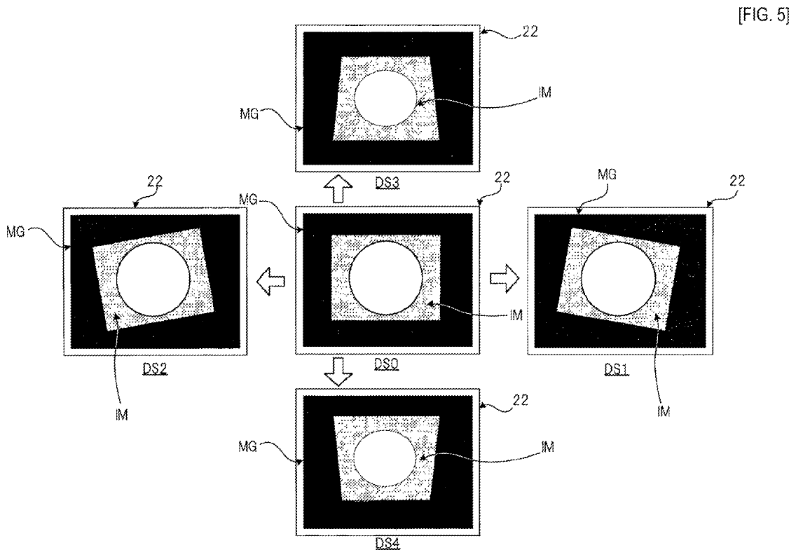

FIG. 5 is a diagram illustrating variations of the display form of the image displayed on the display unit 22. The display form DS0 illustrated in FIG. 5 illustrates a display form in a case where the user's posture satisfies the condition for the attractive posture. In the example of FIG. 5, in a case where the user's posture satisfies the condition for the attractive posture, the image IM smaller than the display area 22A is displayed with a posture (reference posture) determined by the other application program described above or by the user, as illustrated in the display form DS0. The portion of the display area 22A excluding the image IM is the non-display area MG of the image. In the example illustrated in FIG. 5, the reference posture is a posture where two orthogonal sides of the display area 22A and two orthogonal sides of the image IM are parallel.

The display form DS1, the display form DS2, the display form DS3, and the display form DS4 illustrated in FIG. 5 each illustrate a display form for a case where the user's posture does not satisfy the condition for the attractive posture.

The display form DS1 illustrates a display form in a case where the amount of bias in the user's center of gravity to the right direction exceeds the first threshold value. In the display form DS1, the image IM is rotated to the right relative to the display form DS0, changing the display posture of the image IM.

The display form DS2 illustrates a display form in a case where the amount of bias in the user's center of gravity to the left direction exceeds the first threshold value. In the display form DS2, the image IM is rotated to the left relative to the display form DS0, changing the display posture of the image IM.

The display form DS3 illustrates a display form in a case where the amount of bias in the user's center of gravity to the front direction exceeds the second threshold value. In the display form DS3, the image IM is changed to a shape inclined in a depth direction relative to the display form DS0, changing the display shape of the image IM.

The display form DS4 illustrates, for example, a display form in a case where the amount of bias in the user's center of gravity to the back direction exceeds the second threshold value. In the display form DS4, the image IM is changed to a shape inclined in a forward direction relative to the display form DS0, changing the display shape of the image IM.

### Third Example of Changing Image Display Condition

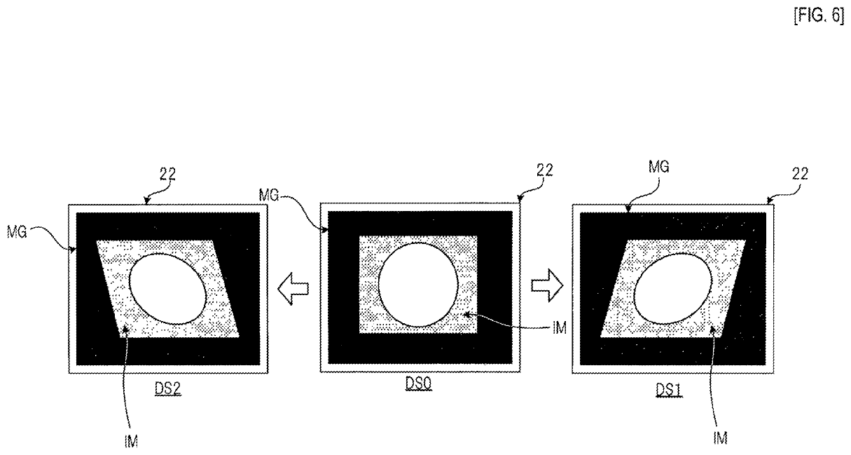

FIG. 6 is a diagram illustrating variations of the display form of the image displayed on the display unit 22. The display form DS0 illustrated in FIG. 6 illustrates a display form in a case where the user's posture satisfies the condition for the attractive posture. In the example of FIG. 6, in a case where the user's posture satisfies the condition for the attractive posture, the rectangular image IM smaller than the display area 22A is displayed at a position (center of the display area 22A) determined by the other application program described above, as illustrated in the display form DS0. The portion of the display area 22A excluding the image IM is the non-display area MG of the image.

The display form DS1 and the display form DS2 illustrated in FIG. 6 each illustrate a display form for a case where the user's posture does not satisfy the condition for the attractive posture.

The display form DS1 illustrates a display form in a case where the amount of bias in the user's center of gravity to the right direction exceeds the first threshold value. In the display form DS1, two sides of the image IM that are parallel to a short side of the display area 22A are inclined to the right direction relative to the display form DS0, changing the display shape of the image IM.

The display form DS2 illustrates a display form in a case where the amount of bias in the user's center of gravity to the left direction exceeds the first threshold value. In the display form DS2, two sides of the image IM that are parallel to the short side of the display area 22A are inclined to the left direction relative to the display form DS0, changing the display shape of the image IM.

### Effect of the Posture Improvement Assistance System 100

As described above, according to the posture improvement assistance system 100, in a case where the posture of the user observing the display unit 22 does not satisfy the condition for the attractive posture, the display form of the image displayed on the display unit 22 is changed from the display form DS0, reducing the visibility of the image, as illustrated in FIG. 4, 5, or 6. As a result, the change in the display form of the image makes the user uncomfortable, and therefore the user improves one's posture and tries to restore the display form of the image to the original form in order to eliminate this discomfort.

Accordingly, the user can be given a strong motivation to improve one's posture, enabling the user to maintain a good posture.

In particular, as in the display forms DS1 and DS2 in FIG. 4, the display forms DS1 to DS4 in FIG. 5, or the display forms DS1 and DS2 in FIG. 6, by linking the direction in which the user's center of gravity is biased with a direction of the change in the display form of the image (the direction of movement of the display position, the direction of rotation of the image, or the direction of deformation of the image), the user can intuitively know which direction to move one's center of gravity to restore the image to the original form. Therefore, compared to a conventional example that simply provides notification of a poor posture with a notification sound, details of the posture to be improved can be intuitively known, and it is possible to help in improving posture.

### Modified Example

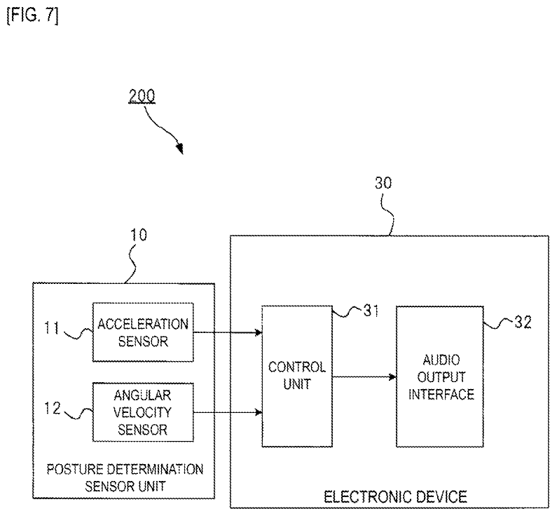

FIG. 7 is a schematic diagram illustrating a schematic configuration of a posture improvement assistance system 200, which is a modified example of the posture improvement assistance system 100 illustrated in FIG. 1. The posture improvement assistance system 200 illustrated in FIG. 7 has an identical configuration as the posture improvement assistance system 100 except that the electronic device 20 has been replaced by an electronic device 30.

The electronic device 30 is, for example, a music player, a radio, a smartphone, a tablet-style terminal, a personal computer, or a television. Specifically, the electronic device 30 includes a control unit 31, an audio output interface 32, and a communication interface (not included in the drawings) for connecting to the posture determination sensor unit 10. The electronic device 30 is used by a user wearing the posture determination sensor unit 10.

The control unit 31 includes, for example, a CPU, a RAM, and a ROM and performs, for example, integrated control of the electronic device 30 in accordance with a program. The ROM of the control unit 31 stores a program that includes the posture improvement assistance program.

The audio output interface 32 is an interface for making a wireless or wired connection to an audio output unit such as stereo ear- or headphones having a right ear earphone and a left ear earphone, a monaural speaker, or a stereo speaker.

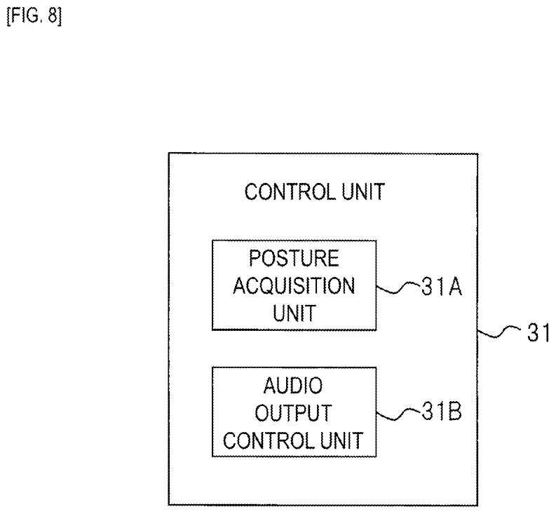

FIG. 8 is a diagram illustrating functional blocks of the control unit 31 of the posture improvement assistance system 200 illustrated in FIG. 7. The CPU of the control unit 31 functions as a posture acquisition unit 31A and an audio output control unit 31B by executing the above-described posture improvement assistance program stored in the ROM. The functions of the posture acquisition unit 31A are identical to the posture acquisition unit 21A in FIG. 2, and thus a description thereof is omitted.

In the electronic device 30, music stored in the ROM can be output from a speaker or earphones, for example, via the audio output interface 32 even in a state where the CPU of the control unit 31 is executing the posture improvement assistance program. Specifically, after attaching the posture determination sensor unit 10 to the user's body and executing the posture improvement assistance program on the electronic device 30, the user can execute an application program for music playback stored in the ROM of the control unit 31 and listen to music using a speaker, earphones, or headphones connected to the audio output interface 32.

The audio output control unit 31B determines whether the user's posture as acquired by the posture acquisition unit 31A satisfies the condition described above (a condition signifying the attractive posture described above) and controls an output condition for sound being output from the audio output unit such as a speaker, earphones, or headphones connected to the audio output interface 32 on the basis of the determination result.

When the audio output unit connected to the audio output interface 32 is stereo earphones or headphones, for example, the “output condition for sound” refers to the balance between the volume of the sound output from the right ear earphone and the volume of the sound output from the left ear earphone (the absolute value of the difference between the two volumes, or the ratio of the two volumes).

In addition, the output condition for the sound may be, for example, the content of sound output from the audio output unit such as earphones or a speaker connected to the audio output interface 32. Specifically, in a case where the sound being output from the audio output unit includes audio from a plurality of audio sources (such as a musical instrument or a human voice), the output condition is a condition that specifies which audio source's audio is to be output or which audio source's audio is to be output at what volume.

In a case where the user's posture as acquired by the posture acquisition unit 31A satisfies the above-noted condition, the audio output control unit 31B outputs sound being output by the music playback application program from the audio output unit, with an output condition determined by the music playback application program or the user (the output condition in this case is defined as the reference state), and in a case where the user's posture does not satisfy the above-noted condition, the audio output control unit 31B outputs the sound with an output condition different from the output condition determined by the music playback application program or the user.

### First Example of Changing Audio Output Condition

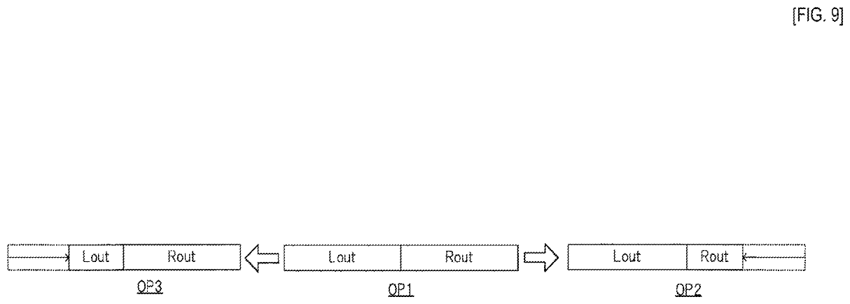

FIG. 9 is a schematic diagram for explaining the content of changes to the output condition made by the audio output control unit 31B illustrated in FIG. 8. FIG. 9 takes as an example a case where the audio output unit includes two audio output units, such as stereo earphones, stereo speakers, or stereo headphones.

An output form OP1 illustrated in FIG. 9 illustrates an output state of sound from the audio output unit in a case where the user's posture satisfies the condition for the attractive posture. An output form OP2 and an output form OP3 illustrated in FIG. 9 each illustrate an output state of the sound from the audio output unit for a case where the user's posture does not satisfy the condition for the attractive posture.

In the output form OP1, a volume Rout of sound output from a first audio output unit for the right ear in the audio output unit and a volume Lout of sound output from a second audio output unit for the left ear in the audio output unit have an identical value, determined by the music playback application program or a user setting. That is, the difference between the volume Rout and the volume Lout is zero (reference value). In other words, the ratio of the volume Rout to the volume Lout (=Rout/Lout) is “1” (reference value).

The output form OP2 illustrates a state when the amount of bias in the user's center of gravity to the right direction exceeds the first threshold value. In the output form OP2, only the volume Rout of the sound output from the first audio output unit is reduced compared to the volume in the output form OP1. That is, the difference between the volume Rout and the volume Lout is substantially less than zero. In other words, the ratio of the volume Rout to the volume Lout (=Rout/Lout) is substantially less than “1.” As a result, the user feels disturbed by changing from a state where the left/right balance of the audio is uniform to a state where the left/right balance of the audio is disrupted.

The output form OP3 illustrates a state when the amount of bias in the user's center of gravity to the left direction exceeds the first threshold value. In the output form OP3, only the volume Lout of the sound output from the second audio output unit is reduced compared to the volume in the output form OP1. That is, the difference between the volume Rout and the volume Lout is substantially greater than zero. In other words, the ratio of the volume Rout to the volume Lout (=Rout/Lout) is substantially greater than “1,” As a result, the user feels disturbed by changing from a state where the left/right balance of the audio is uniform to a state where the left/right balance of the audio is disrupted.

### Second Example of Changing Audio Output Condition

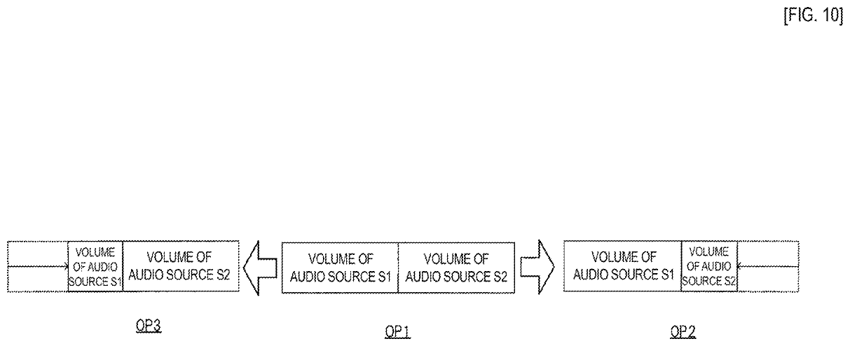

FIG. 10 is a schematic diagram for explaining another example of the content of changes to the output condition made by the audio output control unit 31B illustrated in FIG. 8. FIG. 10 assumes that the sound being output from the audio output unit includes audio from a plurality of audio sources (specifically, music that includes the sound of a musical instrument and singing vocals).

The output form OP1 illustrated in FIG. 10 illustrates the output state of sound from the audio output unit in a case where the user's posture satisfies the condition for the attractive posture. The output form OP2 and the output form OP3 illustrated in FIG. 10 each illustrate an output state of the sound from the audio output unit for a case where the user's posture does not satisfy the condition for the attractive posture.

The output form OP1 is a state where music output from the audio output unit includes audio from all of the audio sources included in the music (audio from an audio source S1 (sound of a musical instrument) and audio from an audio source S2 (vocals)). In addition, audio from the audio source S1 and audio from the audio source S2 are each at a predetermined volume determined by the music playback application program or user settings.

The output form OP2 illustrates a state when the amount of bias in the user's center of gravity to the right direction exceeds the first threshold value. In the output form OP2, only the audio from the audio source S2 (vocals) is changed to a state where the volume is reduced from the predetermined value. As a result, the user feels disturbed by changing from a state where singing and music can be heard together to a state where only the singing is difficult to hear. Note that in the output form OP2, audio from the audio source S2 may be set to non-output (zero volume). This can give the user an additional sense of discomfort.

The output form OP3 illustrates a state when the amount of bias in the user's center of gravity to the left direction exceeds the first threshold value. In the output form OP3, only the audio from the audio source S1 (the sound of a musical instrument, for example) is changed to a state where the volume is reduced from the predetermined value. As a result, the user feels disturbed by changing from a state where singing and music can be heard together to a state where only the music is difficult to hear. Note that in the output form OP3, audio from the audio source S1 may be set to non-output (zero volume). This can give the user an additional sense of discomfort.

### Third Example of Changing Audio Output Condition

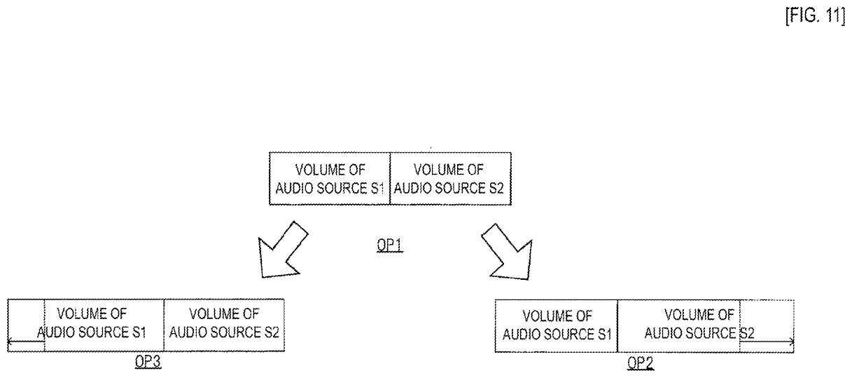

FIG. 11 is a schematic diagram for explaining yet another example of the content of changes to the output condition made by the audio output control unit 31B illustrated in FIG. 8. FIG. 11 assumes that the sound being output from the audio output unit includes audio from a plurality of audio sources (specifically, music that includes sound of a musical instrument and singing).

The output form OP1 illustrated in FIG. 11 illustrates the output state of sound from the audio output unit in a case where the user's posture satisfies the condition for the attractive posture. The output form OP2 and the output form OP3 illustrated in FIG. 11 each illustrate an output state of the sound from the audio output unit for a case where the user's posture does not satisfy the condition for the attractive posture.

The output form OP1 is a state where music output from the audio output unit includes audio from all of the audio sources included in the music (audio from an audio source S1 (sound of a musical instrument) and audio from an audio source S2 (vocals)). In addition, audio from the audio source S1 and audio from the audio source S2 are each at a predetermined volume determined by the music playback application program or user settings.

The output form OP2 illustrates a state when the amount of bias in the user's center of gravity to the right direction exceeds the first threshold value. In the output form OP2, only the audio from the audio source S2 (vocals) is changed to a state where the volume is increased from the predetermined value. As a result, the user feels disturbed by changing from a state where singing and music can be heard together to a state where only the singing is easily heard.

The output form OP3 illustrates a state when the amount of bias in the user's center of gravity to the left direction exceeds the first threshold value. In the output form OP3, only the audio from the audio source S1 (the sound of the musical instrument, for example) is changed to a state where the volume is increased from the predetermined value. As a result, the user feels disturbed by changing from a state where singing and music can be heard together to a state where only the music is easily heard.

### Effect of the Posture Improvement Assistance System 200 According to the Modified Example

As described above, according to the posture improvement assistance system 200, when the posture of a user listening to audio using earphones or a speaker, for example, does not satisfy the condition for the attractive posture, the balance of the audio is disrupted such that only certain sounds become inaudible or only certain sounds become more audible, as illustrated in FIGS. 9, 10, and 11. As a result, the change in audio makes the user uncomfortable, and therefore the user improves one's posture and attempts to restore the output form of the audio to the original form in order to eliminate this discomfort. Accordingly, the user can be given a strong motivation to improve one's posture, enabling the user to maintain a good posture.

In particular, as in the output form OP2 and the output form OP3 in FIG. 9, by linking a direction in which the user's center of gravity is biased with a change in the balance of the audio (audio output unit that reduces volume), the user can intuitively know which direction to move one's center of gravity to restore the audio to the original form. Therefore, compared to a conventional example that simply provides notification of a poor posture with a notification sound, details of the posture to be improved can be intuitively known, and it is possible to help in improving posture.

In addition, as in the output forms OP2 and OP3 in FIGS. 10 and 11, by linking the direction in which the user's center of gravity is biased with the audio of the audio sources changing in volume, the user can intuitively know which direction to move one's center of gravity to restore the audio to the original form. Therefore, compared to a conventional example that simply provides notification of a poor posture with a notification sound, details of the posture to be improved can be intuitively known, and it is possible to help in improving posture.

While various embodiments have been described with reference to the drawings, needless to say, the present invention is not limited to such examples. It will be apparent to those skilled in the art that various changes and modifications can be made within the scope of the claims, and it is understood that these naturally belong within the technical scope of the present invention. Further, each of the components of the above-described embodiments may be combined as desired within a range that does not depart from the spirit of the present invention.

Note that the present application is based on a Japanese patent application (JP 2019-044981) filed Mar. 12, 2019, the contents of which are incorporated herein by reference.

### REFERENCE SIGNS LIST

100, 200 Posture improvement assistance system
10 Posture determination sensor unit
20, 30 Electronic device
21, 31 Control unit
21A, 31A Posture acquisition unit
21B Display control unit
22 Display unit
22A Display area
32 Audio output interface
31B Audio output control unit
IM Image
DS0, DS1, DS2, DS3, DS4 Display form
OP1, OP2, OP3 Output form
MG Non-display area

1. A posture improvement assistance device comprising:
a posture acquisition unit that acquires information on a posture of a user who is looking at an image displayed on a display unit of an electronic device or a user who is listening to sound output from an audio output unit of an electronic device; and
a control unit that, when the information on the posture of the user satisfies a condition that is predetermined and represents a state of proper posture, controls a display condition of the image displayed on the display unit or an output condition of the sound being output from the audio output unit to be in a reference state, and when the information on the posture of the user does not satisfy the condition, controls the display condition or the output condition to be in a state that is changed with respect to the reference state.

2. The posture improvement assistance device according to claim 1, wherein
the display condition relates to a display position of the image, and
when the information on the posture of the user satisfies the condition, the control unit controls the display position of the image displayed on the display unit to be in the reference state as a reference position, and when the information on the posture of the user does not satisfy the condition, the control unit changes the display position of the image to a position different from the reference position.

3. The posture improvement assistance device according to claim 1, wherein
the display condition relates to a display size of the image, and
when the information on the posture of the user satisfies the condition, the control unit controls the display size of the image displayed on the display unit to be in the reference state as a reference size, and when the information on the posture of the user does not satisfy the condition, the control unit changes the display size of the image to a size different from the reference size.

4. The posture improvement assistance device according to claim 1, wherein
the display condition relates to a display posture of the image, and
when the information on the posture of the user satisfies the condition, the control unit controls the display posture of the image displayed on the display unit to be in the reference state as a reference posture, and when the information on the posture of the user does not satisfy the condition, the control unit changes the display posture of the image to a posture rotated from the reference posture.

5. The posture improvement assistance device according to claim 1, wherein
the display condition relates to a display shape of the image, and
when the information on the posture of the user satisfies the condition, the control unit controls the display shape of the image displayed on the display unit to be in the reference state as a reference shape, and when the information on the posture of the user does not satisfy the condition, the control unit changes the display shape of the image to a shape different from the reference shape.

6. The posture improvement assistance device according to claim 1, wherein
the audio output unit comprises a first audio output unit and a second audio output unit,
the output condition relates to a difference or a ratio between a volume of the sound output from the first audio output unit and a volume of the sound output from the second audio output unit, and
when the information on the posture of the user satisfies the condition, the control unit controls the difference or the ratio to be in the reference state as a reference value, and when the information on the posture of the user does not satisfy the condition, the control unit controls the difference or the ratio to be a value different from the reference value.

7. The posture improvement assistance device according to claim 1, wherein
the sound is music that includes audio from a plurality of audio sources,
the output condition relates to a balance of a volume of the audio for each of the audio sources included in the music output from the audio output unit, and
when the information on the posture of the user satisfies the condition, the control unit controls the audio from each of the plurality of audio sources to be in the reference state, the audio being output from the audio output unit at a volume that is predetermined, and when the information on the posture of the user does not satisfy the condition, the control unit changes only the volume of the audio from an audio source, which is a portion of the plurality of audio sources.

8. The posture improvement assistance device according to claim 7, wherein, when the information on the posture of the user does not satisfy the condition, the control unit changes only the volume of the audio from the audio source, which is the portion, to zero.

9. The posture improvement assistance device according to claim 7 wherein, when the information on the posture of the user does not satisfy the condition, the control unit increases only the volume of the audio from the audio source, which is the portion.

10. The posture improvement assistance device according to claim 8, wherein the control unit changes the audio source, which is the portion, in accordance with a direction of bias in a center of gravity of the user based on the information on the posture of the user.

11. A posture improvement assistance method comprising:
a posture acquisition step acquiring information on a posture of a user who is looking at an image displayed on a display unit of an electronic device or a user who is listening to sound output from an audio output unit of an electronic device; and
a control step, when the information on the posture of the user satisfies a condition that is predetermined and represents a state of proper posture, controlling a display condition of the image displayed on the display unit or an output condition of the sound being output from the audio output unit to be in a reference state, and when the information on the posture of the user does not satisfy the condition, controlling the display condition or the output condition to be in a state that is changed with respect to the reference state.

12. A non-transitory storage medium storing a posture improvement assistance program, the program causing a computer to execute:
a posture acquisition step acquiring information on a posture of a user who is looking at an image displayed on a display unit of an electronic device or a user who is listening to sound output from an audio output unit of an electronic device; and
a control step, when the information on the posture of the user satisfies a condition that is predetermined and represents a state of proper posture, controlling a display condition of the image displayed on the display unit or an output condition of the sound being output from the audio output unit to be in a reference state, and when the information on the posture of the user does not satisfy the condition, controlling the display condition or the output condition to be in a state that is changed with respect to the reference state.
